(19) United States
(12) Patent Application Publication
(10) Pub. No.: US 2023/0270711 A1
(43) Pub. Date: Aug. 31, 2023

(54) Improved Anti-Hangover Composition, Its Preparation and Uses

(71) Applicant: Zobrius Pharma AS, Drammen (NO)

(72) Inventors: Bjørn Tore LANGELAND, Tønsberg (NO); Jan KARLSEN, Jar (NO)

(21) Appl. No.: 17/767,633

(22) PCT Filed: Oct. 8, 2020
(86) PCT No.: PCT/EP2020/078322

§ 371 Date: Apr. 8, 2022

(30) Foreign Application Priority Data
Oct. 10, 2019 (NO) 20191208

(51) Int. Cl. A61K 31/26; A61K 36/258; A61K 36/9068; A61K 36/254; A61K 45/06; A23L 33/105; A61P 25/32; A61K 9/00; A61K 31/191; A61K 31/522; A61K 36/77; A61K 31/7004

(52) U.S. Cl. CPC A61K 31/26; A61K 36/258; A61K 36/9068; A61K 36/254; A61K 45/06; A23L 33/105; A61P 25/32; A61K 9/0095; A61K 31/191; A61K 31/522; A61K 36/77; A61K 31/7004

(57) ABSTRACT

A composition for the promotion of alcohol and acetaldehyde degradation in a human is disclosed. The composition, which can be prepared by bringing all the ingredients together in intimate physical admixture, comprises an anti-hangover effective amount of:
(a) essential herbs consisting of Ilex paraguarensis (Yerba mate), Eleutherococcus senticosus (Siberian ginseng), Panax ginseng and Zingiberis officinalis (ginger); and
(b) one or more electrolyte(s) and, optionally,
(c) sulphoraphane.

Preferably, the composition is in the form of a low-volume, aqueous, after-alcohol drink, that can avoid some of the potentially adverse effects of previously-known anti-hangover preparations.

### CROSS REFERENCE TO RELATED APPLICATION

This application is a filing under 35 U.S.C. 371 of International Application No. PCT/EP2020/078322, filed Oct. 8, 2020, entitled “Improved Anti-Hangover Composition, its Preparation and Uses,” which claims priority to Norwegian Patent Application No. 20191208, filed Oct. 10, 2019, which applications are incorporated by reference herein in their entirety.

### FIELD OF THE INVENTION

The present invention relates to a composition useful for promoting alcohol and acetaldehyde metabolism to mitigate unwanted side-effects of alcohol consumption on a human body. Therefore, the composition is effective for treating, preventing or ameliorating over-drunkenness, drunken sickness and hangover, (hereinafter, together, ‘anti-hangover effective’). The composition is preferably in the form of an after-alcohol drink, and comprises anti-hangover-effective herbs or herbal extracts, particularly Yerba mate, together with electrolytes and, optionally, sulphoraphane.

### BACKGROUND TO THE INVENTION

Consumption by a human individual of alcohol may result in an accumulation of acetaldehyde, resulting in one or more of: headache, nausea, shaking and vertigo in the individual. The condition suffered under these circumstances is typically termed a ‘hangover’ or, medically, veisalgia. Fatigue is also often associated with hangover.

A number of products are known in the art, which can be taken to relieve the symptoms of a hangover. Fructose and caffeine mixtures, optionally including vitamin C, are examples of such products. Generally, such products are in powder or tablet form, and are added to water to provide a rehydration product for an individual to drink.

For example, patent specification number GB 2308810 describes a fructose-containing composition for rehydrating or preventing dehydration of an individual, which may therefore inter alia treat symptoms of dehydration, physical exertion or diarrhoea.

The effect of fructose on ethanol metabolism is subject to conflicting opinions, with some studies indicating that fructose alone can stimulate ethanol degradation, but others disagree. The effect of fructose is probably (instead or also) due to its ability to increase ATP turnover, thus making more ADP available for NADH re-oxidation (FIG. 3, hereinbelow). As NADH is a strong inhibitor (Ki=56 μM) of LADH, accelerated NADH re-oxidation may lead to less inhibition of LADH activity. However, it is thought that a dose of at least 20 g (20,000 mg) of fructose needs to be ingested before an anti-hangover effect can be obtained, and often of the order of 90 to 100 g has been employed.

Some anti-hangover products may contain an analgesic, such as paracetamol or aspirin, to provide relief from pain associated with the hangover. Products containing anti-histamines may also be used to combat hangover symptoms. However, it may be undesirable to use analgesics and antihistamines in this manner. For instance, aspirin can exacerbate some of the problems associated with alcohol consumption, such as gastrointestinal bleeding; and using histamine-2 blockers, for example to reduce so-called ‘Asian flushing syndrome’, can escalate alcohol intake and increase the risk of gastro-intestinal cancers and squamous cell carcinoma.

For example, patent specification number WO 98/32434 describes an analgesic composition comprising acetaminophen, aspartic acid and methionine, to alleviate liver toxicity and release hangover as well as to provide an analgesic effect.

Other compositions are known, which comprise mixtures of various herbs and other ingredients.

For example, patent specification number WO 99/61038 discloses a formulation that stimulates a short- and/or long-term psychological feedback, comprising ingredients to be selected from a long list that includes ginseng and ginger. However, this composition is not directed to promoting alcohol metabolism, and also contains several ingredients previously shown to strongly inhibit alcohol metabolizing enzymes and therefore to have the opposite effect from that desired for treating hangover. This is particularly the case for thiol ingredients (e.g. cysteine, acetylcysteine, glutathione, methionine) and also for some of the heterocyclic compounds listed, which inhibit the activity of liver alcohol dehydrogenase (LADH) enzyme (Ki in the μM-mM area).

On the other hand, other publications, such as Japanese patent specification no. JP 0601474, describe a product capable of promoting alcohol metabolism containing, as active ingredients, a glucoside of quercetin, divalent metallic ion and liquorice extract.

One of the present inventors previously developed an alternative and inventive means, disclosed in US patent specification no. 6936283, of suppressing the undesirable effects of excessive alcohol consumption and for treating the onset of symptoms associated with a hangover in a subject. That invention is directed to stimulating specific metallo-enzymes to promote alcohol and acetaldehyde degradation. The means relates to a composition comprising caffeine, Guarana caffeine, Yerba mate, Eleutherococcus senticosus, Panax ginseng, ginger, Glycyrrhiza glabra (liquorice root), Ginkgo biloba (also known as Gingko biloba) and fructose. The inventor's prior composition is thereby free from enzymes and NADH/NAD (nicotinamide adenine dinucleotide), which would reduce the stability of the product. Furthermore, it does not contain analgesics or antihistamines, and provides significantly higher alcohol and acetaldehyde degradation than the fructose-caffeine-vitamin C product previously mentioned.

However, it was pointed out in the US patent specification no. 6936283 that the prior compositions tested still suffered from some significant disadvantages:
1. The composition, when formulated as a drink, occupied a volume of 500 ml in view of the need to accommodate all the ingredients deemed necessary and particularly the weight of fructose (dry powder)—in one example, as much as 90 g—to be incorporated. This is clearly inconvenient for someone to consume when they are already full of alcoholic liquids and, in any case, 500 ml is considered to be a significant volume to ingest in one dose.
2. A modified example that occupied a lesser volume of 200 ml required the omission of the necessary (anti-hangover) amount of fructose to ensure that a solution (aqueous drink) could be formulated. Even 200 ml is not an insignificant amount of liquid to consume. More importantly, it was found that the effectiveness of the resulting fructose-free formulation as an anti-hangover drink was thereby reduced.
3. Moreover, inclusion of high quantities of fructose is known to incur risk to liver function in the consumer, and is therefore contra-indicated as an active ingredient. Ingesting such large amounts of fructose stresses the liver straight away, as fructose is immediately converted by the liver to fructose-1-phosphate (F1P), thereby depleting the liver cells of phosphates.
4. These prior formulations each comprised added caffeine and also Glycyrrhiza glabra (liquorice root), which are also not advisable under the circumstances. For example, substantial amounts of added caffeine (e.g. as caffeine and Guarana caffeine) can result in sleeplessness and/or cardiovascular side-effects, which are generally unwelcome at least. Liquorice ingestion above certain amounts is associated with reduced blood potassium levels, potentially resulting in abnormal heart rhythms, high blood pressure, oedema, lethargy, and other adverse effects.

Subsequent to the disclosure in U.S. Pat. No. 6,936,283, others have attempted to conceive of suitable anti-hangover formulations. These include the disclosures in:
patent specification no. WO 2008/021861, which describes a ginseng, ginger and milk-thistle-containing formulation with optional electrolytes, vitamins, amino acids, and carbohydrates such as high fructose corn syrup. However, no specific formulations are disclosed, nor any clinical data indicating that such compositions have an anti-hangover effect;
patent specification no. WO 2009/094177, which relates to certain ginsenoside compositions in the context of treating or preventing elevated acetaldehyde concentration. There is the suggestion to include other active agents, such as vitamins, anti-oxidants, anti-inflammatories, anti-histamines, glutamine, milk thistle complex and analgesics. An example formulation includes glutamine and silymarin (milk thistle complex) but no clinical tests are included. The focus of this publication is on the ginseng component; and
patent specification no. KR 2017/0085451, which discloses combining ginger with a mixture of either (a) red ginseng, red head and tea germ or (b) bamboo parts and other ingredients to prepare a herbal extract for treating hangover. There no evidence provided that these compositions work in the clinic.

Moreover, none of these patent specifications relate to a herbal composition that contains Yerba mate, so offer no improvement on the original formulation as described in U.S. Pat. No. 6,936,283.

On the other hand, patent specification no. RU 2376027 does include Yerba mate in a ginger- and ginseng-containing formulation. It appears to be a variation of the original formulation in that it relates to a combination of ginger, Eleutherococcus, ginseng, Paullinia cupana (also known as Guarana), holly (Ilex), liquorice, citric acid, succinic acid and Aerosil™.

This specification demonstrates an increase in the rate of elimination of alcohol from the (human) body upon administration of a capsule containing the formulation. However, this fructose-free formulation requires the addition of 360 mg liquorice and 130 mg Paullinia/Guarana (caffeine) per dose, which—as previously mentioned—the present inventors are now trying to avoid (in such quantities). Furthermore, no evidence is provided that such a formulation can be made into an easy-to-take, low volume drink that is palatable enough for consumption as such.

There has therefore been no significant improvement known to the original Yerba mate-containing formulation previously proposed. There has been no solution to the problem of how to formulate an anti-hangover composition comprising a mixture of Yerba mate, ginger and ginseng herbs or herbal extracts that both (i) can be taken as a low volume drink and (ii) can avoid the adverse effects of certain quantities of caffeine, liquorice, fructose and the like.

It is therefore surprising that a solution to these difficulties has now been found. The solution is based on a multi-faceted approach: to substantially increase the amount of Yerba mate, relative to the other essential herbs (ginger, ginsengs), and to add electrolytes. In this way, the amount of fructose can be reduced to sweetener levels, and little or no added caffeine or liquorice is required, yet the anti-hangover effect is still very satisfactory.

Moreover, it has now also been found that the addition of sulphoraphane to the compositions of the invention can provide still further benefits in terms of anti-hangover effects, as will be demonstrated in the examples hereinafter.

The present inventors have therefore made significant improvements to the prior compositions, and thereby provides novel compositions that are surprisingly not only suitable for accelerating alcohol and acetaldehyde metabolism (to ameliorate or prevent alcohol-related damage and hangover symptoms) but also offer certain advantages thereover. These advantages include the ability to formulate an anti-hangover effective composition at low volume (e.g. 50 ml, rather than 500 or 200 ml as in the prior formulations).

### SUMMARY OF THE INVENTION

Therefore, the present invention provides a composition, preferably in the form of a drink, for accelerating—or promoting—degradation of alcohol and acetaldehyde in a human, which composition comprises a combination of ‘essential herbs’ (as defined hereinbelow) and electrolytes, optionally together with sulphoraphane. The composition promotes the activity of aldehyde dehydrogenases and/or prevents, suppresses or reduces other adverse physiological effects of alcohol consumption, such as prevention or amelioration of Asian (sometimes called Japanese) flushing syndrome.

The ‘essential herbs’ component consists of a mixture of Ilex paraguarensis (Yerba mate), Eleutherococcus senticosus (Siberian ginseng), Panax ginseng (Asian ginseng) and Zingiberis officinalis (ginger). These are essential herbal active ingredients having anti-hangover effects in combination.

The one or more electrolytes are preferably selected from sodium, potassium and magnesium (salts). Magnesium is a particularly preferred electrolyte ingredient.

More preferred is when sulphoraphane is also comprised in the composition.

It has surprisingly been found that the compositions of the invention can be formulated as a low-volume, aqueous drink, yet still provide a desired anti-hangover effect. Preferably, the volume of the drink is less than 250 ml, more preferably less than 200 ml and especially preferred is a volume in the range of from about 40 ml to 150 ml, such as 50 ml or 100 ml, particularly 50 ml. Suitably, the volume is less than 150 ml.

Accordingly, the present invention further provides a low-volume drink comprising:
(a) water;
(b) essential herbal active ingredients, consisting of: Ilex paraguarensis (Yerba mate), Eleutherococcus senticosus (Siberian ginseng), Panax ginseng (Asian ginseng) and Zingiberis officinalis (ginger); and
(c) one or more electrolyte(s); and, optionally,
(d) sulphoraphane; and, optionally
(e) one or more additional active ingredient(s); and, optionally,
(f) one or more inert additive(s), selected from carriers, excipients, and the like.

Preferably, the Yerba mate is the major component of the essential herb mixture. More preferably, the Yerba mate comprises at least 30% by dry weight of the essential herbal mixture (component (b)).

Furthermore, the essential herb mixture itself is preferably present in an amount less than about 1000 mg (per dose), although higher amounts can be tolerated.

Unless otherwise specified or the context requires it, the amounts (in μg, mg and g) referred to herein are given relative to a single dose of the composition for administration to an adult human.

Suitably, the composition of the invention is substantially free from added Glycyrrhiza glabra (liquorice root), although a relatively small amount may be present.

Particularly suitable is when the composition is also substantially free from added fructose in excess of an amount required for sweetening the taste (i.e. about 5 g), and is at least substantially free from added fructose in excess of n an amount that provides an anti-hangover effect.

Especially suitable is when the composition is substantially free from added caffeine (any caffeine present as a constituent part of any of the herbal ingredients is excepted. For example, Yerba mate contains caffeine). However, relatively low amounts of added caffeine can be tolerated in the compositions of the invention.

More suitably, the composition is substantially free from each and all of the ingredients in an amount or as added in accordance with the three preceding paragraphs. By this is meant that the composition is substantially free from added liquorice root, fructose and caffeine, or has only minimal amount(s) of each as indicated hereinbelow.

Preferably, the composition comprises ingredients (b) and (c), or (b) and (d), as defined above. More preferably, the composition comprises ingredients (b), (c) and (d), as defined above. Especially preferably, the composition comprises ingredients (b), (c), (d) and (f), as defined above. The composition may alternatively comprise ingredients (b), (c), (e) and (f), or all of (b) to (f). In each case, the composition more preferably also comprises water (ingredient (a)).

More preferably, the electrolyte(s) (c) are selected from alkali metal and alkaline earth metal salts, particularly sodium, potassium and magnesium salts, especially magnesium salts, of suitable organic acids, such as gluconic and lactic acids, e.g. sodium and potassium gluconates and magnesium lactate.

More preferably, the additional active ingredient(s) (e) are selected from other herbs and herbal extracts, such as Ginkgo biloba. Relatively small amounts of Glycyrrhiza glabra (liquorice) may also or alternatively be present.

More preferably, the inert additive(s) (f) are selected from: flavourings, sweeteners, taste-masking agents, colouring agents and aromatic agents (aroma); formulating agents, e.g. stabilising agents, pH modifiers, solvents (e.g. concentrated ethanol), preservatives and buffering agents, (such as citric acid monohydrate); and other excipients, such as potassium sorbate, glycerol, polysorbate 20.

The most preferred formulations of the invention, as described herein, exhibit advantages over the prior art formulations in that they:
1. can be formulated into a relatively low volume drink (50 ml);
2. do not need to contain substantial levels of fructose, which generally avoids fructose's potentially adverse effects on the liver;
3. do not need to contain significant quantities of added caffeine, which generally avoids caffeine's potentially adverse effects to the cardiovascular system;
4. do not need to comprise significant quantities of liquorice, thereby generally avoiding liquorice's potentially adverse effects;
5. do not need the addition of Ginkgo biloba, which (in some people) can contribute to some of the effects of alcohol, such as headaches, and may adversely interact with some drugs (e.g. NSAIDs);
6. do not require such large quantities of essential herb mixture; and
7. are palatable for consumption, yet still have a sufficient anti-hangover effect.

### BRIEF DESCRIPTION OF THE FIGURES

FIG. 1—The basic pathway of ethanol metabolism.

Alcohol is converted to acetaldehyde by three enzymes: alcohol dehydrogenase, catalase and CYP2E1. Acetaldehyde is converted to acetate by aldehyde dehydrogenase.

FIG. 2—Proposed mechanism for stimulation of alcohol and/or acetaldehyde degradation.

Stimulation, due to an activator forming a ternary enzyme-NADH-activator complex being more labile than the normally rate-determining enzyme-NADH product dissociation.
E=enzyme: liver alcohol dehydrogenase or aldehyde dehydrogenase.
O=NAD (oxidized form of the coenzyme)
S=substrate: alcohol or acetaldehyde
P=product: acetaldehyde or acetate
R=NADH (reduced form of the coenzyme)
A=activator: e.g. essential herbs
k3 res=rate-determining step

FIG. 3—Interaction between ethanol metabolism by LADH and mitochondrial respiration.

Ethanol is oxidized to acetaldehyde by LADH (a), with concomitant reduction of NAD to NADH. NADH is then re-oxidized to NAD in the mitochondria (c) by the electron transport chain (d), and oxygen is consumed (g). Substances that are phosphorylated with concomitant formation of ADP from ATP (f) increase electron flux through the respiratory chain (e), with a subsequent increase in oxygen consumption and rates of NADH re-oxidation.

### DETAILED DESCRIPTION OF THE INVENTION

Composition—The present invention provides a composition, suitable for the promotion of alcohol and acetaldehyde degradation in a human, which composition comprises an anti-hangover effective amount of a combination of:
(a) essential herbs consisting of Ilex paraguarensis (Yerba mate), Eleutherococcus senticosus (Siberian ginseng), Panax ginseng (Asian ginseng) and Zingiberis officinalis (ginger);
(b) electrolytes, preferably selected from one or more of sodium, potassium and magnesium salts, especially magnesium; and, optionally,
(c) sulphoraphane.

Further details, regarding each of these and other optional ingredients, follow:

Essential Herbal Ingredients—The essential herbs component consists of four herbal ingredients that are essential in the improved formulation of the invention; they are responsible for stimulating the alcohol and acetaldehyde metabolizing enzymes. The four essential herbal components are: Ilex paraguarensis (Yerba mate), Eleutherococcus senticosus (Siberian ginseng), Panax ginseng (Asian ginseng) and Zingiberis officinalis (ginger).

Nevertheless, the most important of the four herbal components is Yerba mate. It is preferably present as the major component of the essential herb mixture, by dry weight. More preferably, Yerba mate comprises more than 0.1%, such as more than 1%, even more preferably more than 10%. Especially preferred is when Yerba mate comprises at least 30%, such as at least 50%, w/w of the dry herb mixture (component (a)).

Yerba mate is the major essential herb component as its constituent comprise several potentially anti-hangover effective active ingredients, as listed in Table 1:

TABLE 1
Selected constituents of Yerba mate

| | Minimum | Maximum | Average |
|---|---|---|---|
| | amount | amount | amount |
| Moisture | 5.360 | 9.800 | 8.170 |
| Proteins | 8.300 | 13.450 | 10.890 |
| Carbohydrates | 9.700 | 14.180 | 12.040 |
| Starch | 2.560 | 6.630 | 4.550 |
| Glucose | 1.300 | 6.140 | 3.840 |
| Fibers | 14.960 | 19.950 | 16.960 |
| Ashes | 6.310 | 7.780 | 6.910 |
| Chlorine (g) | 0.082 | 0.160 | 0.116 |
| Sulphur | 0.082 | 0.168 | 0.125 |
| Phosphorus (g) | 0.074 | 0.214 | 0.120 |
| Calcium | 0.597 | 0.824 | 0.668 |
| Magnesium (g) | 0.134 | 0.484 | 0.337 |
| Potassium (g) | 1.181 | 1.554 | 1.350 |
| Sodium (g) | 0.000 | 0.003 | 0.002 |
| Iron (mgs) % | — | 94.000 | 59.900 |
| Cuprum (mgs) | 0.600 | 1.600 | 1.260 |
| Manganese (mgs) | 30.200 | 183.000 | 133.180 |
| Caroffin (mgs) | 0.639 | 2.267 | 1.234 |
| Caroffin (vitamin A | 1.065 | 3.779 | 2.095 |
| U.I.) | | | |
| Thiamin (gamma) | 62.300 | 313.100 | 222.700 |
| Riboflavin | 246.000 | 573.900 | 404.300 |
| Ascorbic acid | 8.200 | 20.700 | 11.900 |

Thiamine and riboflavin (B vitamins) are the most interesting components of the Yerba mate from an anti-hangover-effective standpoint.

Studies (e.g. those by Heck and de Mejia, reported in J. Food Sci. (2007) 72(9) R138-151; and Burris et al in Chilean Journal of Agricultural Research (2012), 72(2), 268-274) show that Yerba mate has many other constituents, including caffeine at 1-2% of dry weight, although other sources indicate as little as 0.4% or as much as 2.4% (by dry weight of Yerba mate).

The essential herbs, taken together, are preferably present in an anti-hangover-effective amount in the composition.

One of the advantages of the present invention is that a lower overall weight of essential herbs can provide a satisfactory anti-hangover formulation, compared to previously-known compositions. For example, the total weight of essential herbs can be in the range of from 100 to 1000 mg (although more is possible), such as 300 to 600 mg, e.g., 400 to 450 mg (per dose).

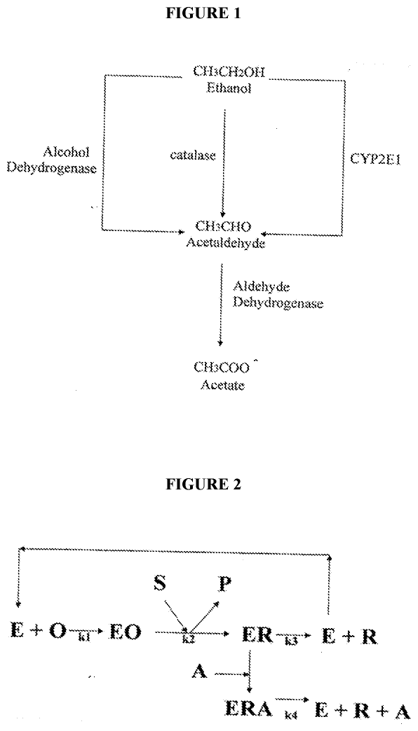

Although not wishing to be bound by theory, the present inventors suggest that the effect of the composition of the invention on stimulation of ethanol degradation and prevention of hangover is probably due to active components of the essential herbal ingredients forming ternary enzyme-NADH-activator (ERA) complexes that are more labile than the normally rate-limiting enzyme-NADH (ER) dissociation or by protecting catalytic residue(s) from oxidation. The basic pathways of ethanol metabolism are shown in FIG. 1. The proposed mechanism is similar to that known for 2,2′- and 4,4′-bipyridine. The accelerated alcohol degradation may also in part be due stimulation of catalase and/or CYP2E1, which are also involved in alcohol metabolism.

As described in Kitson. T. M. (1977) Journal of Studies on Alcohol. 38, 96-113, and in Langeland, B. T. (one of the present inventors) and McKinley-McKee, J. S., (1996) Alcohol & Alcoholism. 31, 75-80, accumulation of acetaldehyde is the main cause of hangover symptoms. The effect of the composition of the invention on hangover also indicates a stimulation of aldehyde dehydrogenase activity. The necessary increase in acetaldehyde degradation may also in part be covered by stimulated LADH, which is also capable of aldehyde oxidation.

Electrolytes—The electrolytes in the composition, as described hereinabove, reduce or prevent dehydration caused by the diuretic effect of alcohol.

The electrolytes are preferably selected from alkali metal and alkaline earth metal ion, particularly sodium, potassium and magnesium. More preferably, all three are present in the composition of the invention. Especially preferred is when the electrolyte component comprises magnesium in a major proportion, or when potassium is a major part of the electrolyte component.

As a guide, the weight of electrolytes per dose (suitable for use in a 50 ml aqueous formulation) can suitably be included in amounts relative to and in the order of the following, per ion: sodium ion, 10-50 mg, e.g., about 20, such as 20.02 mg; potassium ion, 200-500 mg, e.g., about 300 mg, such as 300.44 mg; magnesium ion, 40-150 mg, e.g. about 60 mg, such as 66.03 mg.

For example, the weight of electrolytes (suitable for use in a 50 ml aqueous formulation) can suitably be included in amounts relative to and in the order of the following, per ion: sodium ion, 20.02 mg; potassium ion, 300.44 mg; magnesium ion, 66.03 mg.

Preferably, the electrolyte(s) are comprised in the formulation in the form of alkali metal and alkaline earth metal salts of organic or inorganic acids, particularly sodium, potassium and magnesium salts, especially potassium and/or magnesium salts, of suitable weak acids, such as weak organic acids.

Suitable organic acids for this purpose include gluconic and lactic acids, although others suitable for human consumption may be used and are known to those skilled in the art.

Preferably, the electrolyte(s) are selected from one or more of: sodium gluconate, potassium gluconate and/or magnesium lactate.

Sulphoraphane—Sulphoraphane, or 1-isothiocyanato-4-methylsulphinylbutane, is a natural plant compound found in many cruciferous vegetables, such as broccoli, cabbage, cauliflower and kale. It is produced from glucoraphanin, a glucosinolate, by the enzyme myrosinase.

One of the advantages of including sulphoraphane in the composition of the present invention is to increase the activity of usually inactive/low activity aldehyde dehydrogenases like hsALDH (human salivary aldehyde dehydrogenase). It may also increase the activity of the naturally-occurring variant of ALDH2 found in approximately 30-50% of the East Asian population (in Japan, Korea and China), which reduced activity is the cause of so-called Asian flushing syndrome.

Although not wishing to be bound by theory, sulphoraphane is believed to work with the other herbal ingredients by protecting from oxidation catalytic residue(s) in both ALDH2 variants. This leads to an increase in catalytic efficiency and hence activation or stimulation of the enzymes.

It is believed that this is the first time that sulphoraphane has been proposed for use as an anti-hangover agent in a composition to promote alcohol and acetaldehyde degradation in a human that comprises (any of) the essential herbs of the present invention.

Accordingly, the present invention further provides a composition for the promotion of alcohol and acetaldehyde degradation in a human, which composition comprises an anti-hangover effective amount of a combination of sulphoraphane, and one or more of the essential herbs (as defined hereinbefore), and optional other ingredients (such as electrolytes, additional active ingredients and inert additives, as further defined herein).

Preferably, the sulphoraphane-containing composition comprises (i) Yerba mate and optionally other essential herbs (as defined hereinbefore) and/or (ii) electrolytes selected from those herein described as preferred. More preferably, the sulphoraphane-containing composition comprises the essential herb mix (as defined herein), electrolytes (such as alkali and/or alkaline earth metal ions, particularly as salts of weak acids, e.g. potassium and sodium glutamates and magnesium lactate), optionally together with other ingredients as further defined herein.

Sulphoraphane may be present as an active ingredient in an amount that does not exceed that capable of maintaining storage stability (with respect to decomposition of the sulphoraphane to odorous sulphur-containing degradation products) for the required storage time (shelf life).

Suitably, therefore, sulphoraphane may be present as an active ingredient in an amount in the range of from 200 to 800, preferably around 300 to 600 μg per dose. For example, sulphoraphane may be present as an active ingredient in an amount in the range of from 260 to 600 μg per dose, preferably around 300 to 450 e.g. 400 μg per dose.

Sulphoraphane may be present in a ratio in the range of from 0.005 to 0.05, e.g. 0.03 parts sulphoraphane to 1 part essential herbs; preferably, any sulphoraphane is present in about a 1:0.05 ratio to the essential herbs.

Sulphoraphane is often available as a standardised concentrate of broccoli or broccoli sprouts having a sulphoraphane concentration of around 0.4 to 5% (w/w). The amount of concentrate added to the formulation of the invention needs to be adjusted to deliver the correct amount of sulphoraphane. For example, 8 mg of a 5% concentrate would be needed to deliver 400 μg of sulphoraphane via the formulation, per dose.

Additional Active Ingredients—Additional active ingredients may optionally be present in the composition in order to boost or complement the anti-hangover effect of the essential active ingredients. It is preferred that any active ingredients are natural products or extracts of these, as opposed to synthetic chemicals or pharmaceutical drugs. Other herbs may therefore be added, but in relatively low amounts compared to the essential herbs. It is preferred when the only active herbal ingredients in the formulation of the invention are the essential herbs, as defined hereinbefore.

For example, Ginkgo biloba may also be included (e.g. from 50 to 250 mg per dose or in an amount equivalent to 0.25 to 0.75 parts G. biloba:1 part essential herbs), which may contribute positive anti-hangover effects on brain function and blood circulation.

As a further example, up to about 250 mg, preferably not more than 100 mg per dose, preferably below about 20 mg, e.g. around 10 mg of Glycyrrhiza glabra (liquorice root) may be added.

Nevertheless, especially preferred is when the formulation is substantially free from Ginkgo biloba; and/or comprises only relatively low amounts of Glycyrrhiza glabra (liquorice) in the range of 0-20 mg.

Other ingredients may also have a beneficial anti-hangover effect, such as green tea, and B-vitamins e.g. riboflavin and thiamine.

Additional active ingredients are preferably of natural origin and are extracts from products of nature, particularly plant products, or their synthetic equivalents.

It is also possible to add analgesics, antimicrobials or other pharmacologically active ingredients to the composition, although it is preferred that the formulation comprises only herbs or herbal extracts, vitamins and minerals as additional active ingredients.

In relatively small amounts, it is also possible to include added caffeine—either as caffeine itself or, for example, in the form of Guarana caffeine. However, as described above, caffeine is also one of the known constituents of the essential herb mixture (Yerba mate), so it is preferred not to add caffeine above about 3 mg. More preferably, the amount of added caffeine should be less than 1 mg, such as about 0.0 to 0.5 mg.

Inert Additives—The composition of the present invention may optionally also contain one or more inert additives, such as: solvents for any of the ingredients (e.g. just enough 95% ethanol or glycerol to solubilise the herbal ingredients or extracts); additional sugars (including small amounts of fructose as described below, e.g. 5 g per dose) or other sweeteners (e.g. glycerol, aspartame, acesulfame K, sucralose, saccharin, neotame and polydextrose); flavourings, including taste-masking agents (e.g. citric acid monohydrate); colouring agents; vitamins; stabilising agents (e.g. emulsifiers or solubilisers, such as polysorbate 20); pH modifiers, buffering agents and preservatives (e.g. potassium sorbate, sodium benzoate, citric acid monohydrate, phosphoric acid); aromatic agents (aroma) and the like.

Some of the inert additives may have more than one function in the formulation.

It is understood that about 20 g or more fructose is needed to have a significant anti-hangover effect; some prior art formulations include about 90 g—giving rise to the need for a large volume of liquid to make up a drink. It has now been found that, using a specific combination of essential herbs and including electrolytes and/or sulphoraphane, it is not necessary to include these large amounts of fructose. Accordingly, for sweetening or taste-masking, the compositions of the present invention may comprise up to about 20 g of fructose, but preferably in the region of about 5 to 11 g, such as about 3 g to 6 g, (e.g., 4 or 5 g).

The composition may include fruit flavourings and/or whole fruit powder, such as orange, or mixed citrus such as lemon and lime, particularly when in the form of a drink.

The composition, when dissolved in water (such as tap water or still water) or other suitable liquid, can be made effervescent, typically by carbonation e.g. by adding carbon dioxide. Alternatively, naturally effervescent spring water could be used. Accordingly, water is a preferred inert additive.

Ingredient Parameters—Any additional ingredient or additive (i.e. active or inert) should not be present in an amount that reduces the effect of the composition by either competing with the essential herbal ingredients for binding to the active site of the target enzyme or by causing inhibition or inactivation thereof Δny small reduction in effect observed in practice should not be significant (with respect to the total composition).

Furthermore, ‘inert’ ingredients do not have a therapeutic effect and nor do they contribute to any of the anti-hangover effects mentioned herein. They are primarily to ensure that the formulation can be prepared, stored and palatably consumed.

Some ingredients may perform more than one function in the formulation of the invention.

It is especially preferred when the improved formulations of the present invention do not comprise (i.e. are substantially free from) any one of the following added ingredients: fructose in an amount significantly exceeding that required to act as sweetener (i.e. approx. 4-5 g per dose) and anyway less than about 20 g; caffeine (including Guarana caffeine) in an amount exceeding about 3 mg; and/or G. glabra (liquorice root) in an amount exceeding about 20 mg, such as less than 10 mg and preferably not exceeding about 5 mg.

Accordingly, the present invention further provides a composition as defined herein when: substantially free from hangover-effective amounts of fructose; substantially free from added caffeine; and/or substantially free from G. glabra. For clarity, this applies equally in respect of caffeine whether in the form of Guarana caffeine or other caffeine-containing herbs than the essential herbs.

Preferably, the composition of the present invention is substantially free from all three ingredients, combined (added caffeine, hang-over effective amounts of fructose and added liquorice root).

As mentioned before, particularly preferred compositions of the invention comprise, as essential herb ingredients, relatively low amounts of ginger and ginseng, and relatively high amounts of Yerba mate, based on the total dry weight of the essential herb component.

Suitably, the essential herb component comprises 150-500 mg Yerba mate and 100-300 mg of the other three essential herbs, combined. In this case, the dry weight of the Yerba mate may comprise in the range of from about 0.5 to 5 times (i.e. 50 to 500% of) the combined dry weight of the other essential herb ingredients. However, higher amounts of the essential herbs may be used, such as up to an overall weight about 1800 mg, wherein the Yerba mate component preferably comprises at least 30% of the essential herb mixture.

The amount of each component or ingredient in the composition is independently selected, depending on the particular target individual consumer and/or on the particular use(s) to which the composition, component or ingredient is to be put.

The composition preferably comprises, per dosage form (preferably, per 50 ml aqueous composition for drinking):
(a) as essential herbal ingredients: 150-500 mg Ilex paraguariensis, 30-100 mg Panax Ginseng, 20-50 mg Eleutherococcus senticosus and 50-150 mg Zingiberis officinalis; and
(b) as electrolytes: 150-400 mg sodium gluconate, 1500-3000 mg potassium gluconate, and 500-1100 mg magnesium lactate, or the equivalent amounts of the Na, K and Mg ions when using alternative salts.

Optionally and more preferably, the composition may further comprise (c) 200-800 μg sulphoraphane.

Optionally, the composition may comprise (d) small quantities, as necessary, of inert ingredients (e.g. sweeteners, flavourings, colourings, solubilisers, emulsifiers, stabilisers, whole fruit powder and the like as hereinbefore described).

In a preferred embodiment of the present invention, the composition comprises, per dosage form (preferably, per 50 ml aqueous composition for drinking):
(a) as essential herbal ingredients: 150-500 mg Ilex paraguariensis, 30-100 mg Panax Ginseng, 20-50 mg Eleutherococcus senticosus and 50-150 mg Zingiberis officinalis;
(b) as electrolytes: 150-400 mg sodium gluconate, 1500-2500 mg potassium gluconate, and 500-1000 mg magnesium lactate, or the equivalent amounts of the Na, K and Mg ions when using alternative salts;
(c) optionally and more preferably, 200-400 μg sulphoraphane;
(d) optionally and more preferably, small quantities, as necessary, of inert ingredients (e.g. sweeteners, flavourings, colourings, solubilisers, emulsifiers, stabilisers, whole fruit powder and the like).

Optionally, small quantities of additional active ingredients, such as caffeine, Ginkgo biloba and/or Glycyrrhiza glabra (liquorice root) may be included, as previously indicated.

Percentages by weight of the total dry ingredients of the improved formulation of this invention may be any found suitable within the guidelines given hereinbefore, such as:
(a) as essential herbs: Yerba mate (0.5-5.0%, preferably 2.96-3.9, e.g. 3.6%), Eleutherococcus senticosus (0.05-2.0%, preferably 0.32-0.42, e.g. 0.39%), Panax ginseng (0.01-2.0%, such as 0.01-1.0%, preferably 0.62-0.76%), ginger (0.3-3.0%, preferably 0.98 to 1.2%);
(b) (0.3-6.0%, such as 0.3-3.0%, preferably 2.24 to 2.73%) sodium gluconate, (5.0-43.0%, such as 5.0-30.0%, preferably 21.0-25.8%, such as 21.20-22.0%) potassium gluconate, (3.0-15.0%, such as 3.0-10.0%, preferably 6.48-7.90%) magnesium lactate, or the equivalent amounts of the Na, K and Mg ions when using alternative salts;
(c) optionally and more suitably, (0.00-0.3%, such as 0.03-0.3%, preferably 0.05-0.1%, more preferably about 0.06%) of sulphoraphane;
(d) optionally, additional active ingredients, e.g. Ginkgo biloba (0.00-3.0%, preferably 0.16-1.77%) and/or added caffeine (0.00-1.0%, preferably 0.01-0.1%); and
(e) optionally, additional inert ingredients, e.g. sweeteners, flavourings, colourings, solubilisers, emulsifiers, stabilisers, whole fruit powder and the like as hereinbefore defined (when present, 1-75%, such as 1-65%, preferably 57.4-61.5%).

For example, percentages by weight of the total dry ingredients of the improved formulation of this invention may be any found suitable within the guidelines given hereinbefore, such as:
(a) as essential herbs: Yerba mate (0.5-5.0%, preferably 2.96%), Eleutherococcus senticosus (0.05-2.0%, preferably 0.32%), Panax ginseng (0.01-1.0%, preferably 0.62%), ginger (0.3-3.0%, preferably 0.98%);
(b) (0.3-3.0%, preferably 2.24%) sodium gluconate, (5.0-30.0%, preferably 21.20-22.0%) potassium gluconate, (3.0-10.0%, preferably 6.48%) magnesium lactate, or the equivalent amounts of the Na, K and Mg ions when using alternative salts;
(c) optionally and more suitably, 1.3-2.57%, preferably 1.93% of sulphoraphane; (d) optionally, additional active ingredients, e.g. Ginkgo biloba (0.00-3.0%, preferably 1.77%); and
(e) optionally, additional inert ingredients, e.g. sweeteners, flavourings, colourings, solubilisers, emulsifiers, stabilisers, whole fruit powder and the like (1-65%, preferably 61.5%).

A particularly preferred ratio of each of the essential herbs of the present formulation to each other is 10:2:1:3 for Ilex paraguariensis:Panax ginseng:Eleutherococcus senticosus:Zingiberis officinalis, respectively.

A particularly preferred ratio of each of the electrolytes of the present formulation to each other is 1:10:3 for sodium (Na+), potassium (K+) and magnesium (Mg++) ions, respectively.

Preparation

Liquid Compositions—The dry ingredients are preferably dissolved in water or other suitable liquid to provide an aqueous liquid composition. Preferably, the mixture of dry ingredients is dissolved in a ratio of 1 part mixture of dry ingredients to in the range of from 2 to 50 parts water/suitable liquid, such as 3 to 10 parts water/liquid; and, more preferably, 1 part mixture of dry ingredients to 5 parts water/liquid.

Typically, a preferred amount for consumption in one dose by an individual is between 20 to 200 ml of the aqueous liquid composition; more preferably, 50 to 100 ml, especially a 50 ml ‘shot’. Smaller volumes of the liquid composition (e.g. a concentrate, volume suitably about 20 to 30 ml, e.g. 25 ml) may be further diluted to the desired volume for consumption with water or other compatible liquid, depending on consumer preference and convenience. The pH of the aqueous composition is preferably about 3.0 to 7.5, more preferably 5.0 to 5.5.

A method for preparing an aqueous liquid composition, such as an after-alcohol drink, of the invention suitably comprises:
(a) mixing together all the dry ingredients, including the essential herbal ingredients, to form a dry mixture;
(b) adding enough aqueous solvent to enable the mixture to form a concentrate;
(c) stirring the solvent/herb mixture provided by step (b) to form the concentrate;
(d) adding any remaining ingredients and mixing until homogeneous to form a final concentrate; and, optionally,
(e) diluting the final concentrate produced by step (d) with an aqueous liquid until the desired volume is obtained.

For example, a method is provided for preparing an aqueous liquid composition of the present invention, which method comprises:
(a) mixing all the dry ingredients together to form a dry mixture;
(b) adding water to the mixture provided by step (a) in a ratio of in the range of from 1:2 to 2:4 of dry mixture:water;
(c) stirring the mixture provided by step (b) until homogenous to form a concentrate;
(d) adding any remaining ingredients, such as the electrolytes, and any additional active ingredients and inert ingredients, including preservatives, colouring, flavouring, effervescing and/or stabilising agents; and
(e) optionally diluting the concentrate produced in step (d) until a desired volume is obtained.

Alternatively, the method may comprise:
(a) mixing the four essential herbal ingredients (Yerba mate, Panax ginseng, ginger and Siberian ginseng (Eleutherococcus senticosus)) together to form a dry mixture;
(b) adding just enough ethanol to the mixture to dissolve the four ingredients (to a final concentration of 0.67% in the finished 50 ml product);
(c) stirring the mixture provided by step (b) until homogenous to form a concentrate;
(d) adding enough water in which to add and dissolve the remaining ingredients.
(e) adding any remaining ingredients, including preservatives, colouring, flavouring, effervescing and/or stabilising agents to produce a final concentration; and
(f) diluting the concentrate produced in step (e) until the desired volume is obtained.

Solid or Dry Compositions—As well as being suitable for formulating, using methods known to those skilled in the art, as a solution, syrup or other liquid form (such as a beverage or drink or shot), the composition may also be formulated as a solid, such as a dry powder, granules or granulates and tablets (including effervescent and melt tablets).

The composition of the present invention may alternatively be formulated as a pharmaceutical preparation. Where appropriate, this pharmaceutical preparation additionally comprises a pharmaceutically acceptable carrier. Suitable carriers and the formulation of such pharmaceuticals are known to persons skilled in the art.

Likewise, the composition of the present invention may alternatively be formulated as a nutriceutical preparation. Where appropriate, this nutriceutical preparation additionally comprises a nutriceutically acceptable carrier. Suitable carriers and the formulation of such nutriceuticals are known to persons skilled in the art.

The present invention further provides a process for preparing a solid or dry composition of the invention, which process comprises bringing all the ingredients together in intimate physical admixture.

Presentation and Packaging—The final form of the composition may be packaged in a container such as a bottle, blister pack, stick pack or sachet, which may optionally itself be further packaged in outer packaging such as a box, together with instructions for use of the composition. Preferably, the container is a bottle, made from glass or a polymeric material. Especially where there is no outer packaging, the instructions for use may be evident from a label or other indication affixed to or integral with the container.

The instructions for use where the composition is in liquid form may indicate that the container should first be shaken before consumption. Preferably, the composition of the invention will be consumed after ending alcohol consumption. More preferably, the composition is in a form that permits taking one dose per day (when required). Especially preferred is when the composition is in the form of a single, 50 ml aqueous liquid for drinking after consuming alcohol.

Preferred aspects of the methods, doses and types of preparation as described above correspond to the preferred aspects of the compositions as set out hereinbefore.

Uses—The composition of the present invention finds application, for example, in reducing or preventing the onset of symptoms associated with hangover in an individual. The composition may also be taken to obtain lower alcohol levels in the blood the day after alcohol consumption. The composition will also in part reduce or prevent dehydration.

In the case of preventing the onset of symptoms associated with hangover, the composition can be taken immediately after alcohol consumption. By accelerating the key enzymes of alcohol metabolism, alcohol and acetaldehyde degradation will increase and suppress the undesirable effects of excessive alcohol consumption (e.g. headache, cardiovascular disorders, thirst, nausea, shaking, vertigo, fatigue and coordination difficulties).

The present invention therefore provides a composition suitable for use in one or more of:
(a) reducing and/or preventing the onset of symptoms associated with hangover;
(b) accelerating alcohol degradation;
(c) reducing and/or preventing dehydration; and
(d) accelerating acetaldehyde degradation

The composition of the present invention may also be taken to provide an increased energy level in an individual after physical exertion due to exercising or illness. Individuals travelling for long periods of time, such as on long haul aircraft flights can also become exerted and dehydrated, particularly due to alcohol consumption, and may therefore also benefit from taking the composition.

The composition of the present invention may in addition be suitable for use in one or more of:
(a) the treatment of acute alcohol poisoning;
(b) increasing the rate of alcohol and acetaldehyde degradation, and thereby depressing cell and tissue damages caused by these highly reactive reagents; and
(c) depressing the craving for alcohol in alcoholics.

If the acceleration of alcohol degradation in a particular consumer exceeds the acceleration of acetaldehyde degradation, the present invention could also find applications in enhancing the effect of drugs, like disulfiram, which (due to their sulphur content) tend to inhibit ALDH enzymatic action. Then, by reduction of the needed drug dose, side effects and drug toxicity may be depressed or removed.

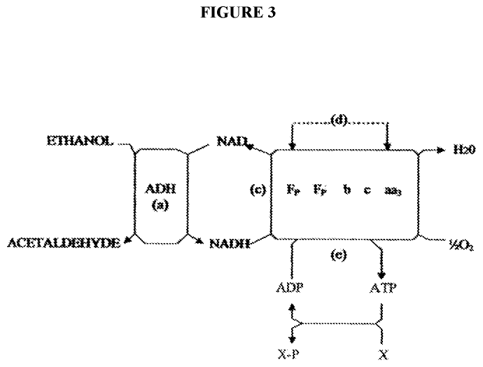

The present invention therefore provides a composition as defined herein, suitable for use in the promotion of alcohol and acetaldehyde degradation in a human, optionally (a) to suppress the undesirable effects of excessive alcohol consumption and/or (b) to treat the onset of symptoms associated with a hangover in a subject and/or (c) to bind to acetaldehyde dehydrogenase in a human. This is illustrated in FIG. 2 herein, showing the binding to the ALDH enzyme (leading to (b)) and formation of an enzyme-NADH-Composition complex that dissociates faster than the normally rate-limiting enzyme-NADH dissociation step.

The present invention further provides a composition as defined herein for use in the prevention, treatment or amelioration of: any one or more or: Japanese or Asian flushing syndrome; alcohol flush reaction; the symptoms associated with either of the foregoing; and to increase the activity of an aldehyde dehydrogenase such as human salivary aldehyde dehydrogenase (hsALDH) in a human.

Alcohol flush reaction (AFR) is a condition in which a person develops flushes or blotches associated with erythema on the face, neck, shoulders, and (in some cases) the entire body after consuming alcoholic beverages. Asian flushing syndrome includes facial flushing, nausea, headaches and a fast heart rate.

Further uses of the composition of the invention include uses in the methods of treatment described below.

Methods of treatment—Accordingly, the present invention further provides a method for promoting alcohol and acetaldehyde degradation in a subject, comprising administering to said subject an effective amount of a composition according to the invention.

The present invention still further provides a method for the promotion of alcohol and acetaldehyde degradation to suppress the undesirable effects of excessive alcohol consumption and/or for treating the onset of symptoms associated with a hangover, which method comprises administering to a subject in need thereof an effective amount of a composition according to the invention.

It will be understood that any of the uses of the composition of the invention outlined hereinbefore can be adapted to a method of treatment in like manner. For example:
the present invention further provides a method for the treatment of hangover resulting from alcohol consumption in a subject, which method comprises administration to the subject of an anti-hangover-effective amount of a composition of the invention, as described hereinabove; and
the present invention further provides a method for the treatment of symptoms resulting from alcohol consumption in a subject, such as one or more symptoms selected from the group consisting of: headache, nausea, dehydration, fatigue, shaking and vertigo, which method comprises administration to the subject of an anti-hangover-effective amount of a composition of the invention, as described hereinabove.

The present invention further provides an improved composition for the promotion of alcohol and acetaldehyde degradation and/or for the relief of hangover symptoms in a subject, which composition comprises
(i) a mixture of herbs consisting of Yerba mate, Eleutherococcus senticosus, Panax ginseng and ginger; and, optionally,
(ii) one or more of an additional ingredient selected from Glycyrrhiza glabra, Ginkgo biloba and fructose,
wherein the improvement is selected from the group consisting of:
(a) the Yerba mate comprises a minimum of 0.1%, such as more than 1%, preferably more than 10%, e.g. more than 30 to 50%, of the mixture of herbs as defined in (i);
(b) the caffeine and Guarana are each absent or are present in an amount up to 10 mg (i.e. 0-10 mg, per dose);
(c) the fructose is either absent or is present in an amount less than that having a significant physiological effect on alcohol degradation, such as an amount in the range up to 20 g, preferably from 0 to 10 g, such as from 4 to 5 g (per dose);
(d) the Ginkgo biloba either absent or is present in an amount less than 100 mg, preferably less than 20 mg, such as 0 to 10 mg (per dose);
(e) the Glycyrrhiza glabra is present in an amount less than 250 mg, such as less than 100 mg, preferably less than 20 mg, such as 0 to 10 mg (per dose);
(f) the composition further comprises electrolyte(s), such as 20-50 mg sodium ion, 300-500 mg potassium ion, and 60-150 mg magnesium ion (per dose); and
(g) the composition optionally further comprises sulphoraphane, such as 200 to 800 μg per dose.

Preferably, the composition of the invention is one wherein the improvement is a combination of each of (a), (c) and (f) as stated above. More preferably, the improvement is a combination of each of (a), (c), (d) and (f) as stated above. Especially preferred is when the improvement is a combination of each of (a) to (f) and optionally (g), as stated above. Also preferred is wherein the improvement is any combination of (a) to (0, as stated above together with (g), as stated above.

### EXAMPLES

The present invention will now be illustrated by the following non-limiting Examples.

Clinical Study Protocol—The studies presented in Examples 1 and 2 were carried out with 10 individuals (6 males and 4 females, aged 24-57 years).

In the control study (using water alone as the after-alcohol drink), the test persons were given 8 oz. (ca.2.4 dl) of vodka mixed with 6 dl Sprite® (The Coca Cola Company). The alcohol was consumed within one hour. At a given time, 30-60 minutes after alcohol consumption, each person drank a given volume of water (control composition). The alcohol level was then measured as described below. The results are provided in Tables 2 and 3.

One week later, a second study was carried out, wherein the same test persons were given the same amounts of vodka and Sprite®, respectively. At the given time after alcohol consumption, each test person drank the given volume of a test composition. The alcohol level was then measured as described just below.

Blood alcohol concentration (BAC) was measured, using a CA 2000 Digital Alcohol Detector, once every 30 minutes in each experiment. Each value is an average of four parallel measurements. Evaluation of effect against hangover was graded on a scale from 1-10 (where 10 represents the greatest effect).

### Example 1: Comparison of a Prior Art (500 ml Fructose (90 g)/Caffeine/G. glabra) Formulation with a Formulation of the Invention (500 ml)

The study protocol was carried out as described above, wherein the test composition comprised Comparative Composition A (defined below); the given time was 45 minutes; and the given volume was 500 ml. The same parameters can be repeated, but using a test composition according to the invention (Composition 1, defined below), and the results compared with those for Comparative Composition A.

Comparative Composition A—A prior art formulation, specifically disclosed in US patent specification no. 6936283 (column 6, ll. 45-50), was prepared, comprising: 35 mg caffeine, 28 mg Guarana caffeine, 27 mg Yerba mate (Ilex paraguariensis), 360 mg Eleutherococcus senticosus, 70 mg Panax ginseng, 360 mg Glycyrrhiza glabra (liquorice root), 1 g ginger and 90 g of fructose.

Natural orange flavouring was added, and the composition was diluted to a total volume of 500 ml with water. The solution was then carbonated to provide Comparative Composition A.

Composition 1 of the invention—Composition 1 (a formulation of the invention) comprises: 250 mg Yerba mate (Ilex paraguariensis), 53 mg Panax Ginseng, 27 mg Eleutherococcus senticosus, 83 mg Zingiberis officinalis, 400 μg sulphoraphane, 300 mg sodium gluconate, 3000 mg potassium gluconate and 1100 mg magnesium lactate.

Results—When taken 45 minutes after alcohol consumption, the prior art comparative composition A was found to accelerate alcohol degradation and reduce or prevent the symptoms associated with a hangover, compared to the control (water). The results from this first study (on the prior art formulation) are shown in Table 2. The increase was between 67 and 92%, with an average of 83%. Hangover symptoms, as graded on a scale from 1-10 (equal to 10-100% reduction in hangover symptoms) were also markedly (80-100%) reduced.

TABLE 2
The effect of a [500 ml, aqueous] 90 g fructose-containing prior art
composition on alcohol metabolism and hangover symptoms

| | Alcohol metabolism (%/hour) | | | | | |
|---|---|---|---|---|---|---|
| | | | | | Prior art | Effect |
| | | | Weight | Base- | Composition | against |
| Individual | Sex | Age | (kg) | line | A | hangover |
| 1 | Male | 40 | 70 | 0.15 | 0.28 | 8 |
| 2 | Male | 35 | 95 | 0.15 | 0.25 | 10 |
| 3 | Male | 29 | 85 | 0.16 | 0.3 | 10 |
| 4 | Male | 57 | 115 | 0.17 | 0.3 | 8 |
| 5 | Male | 30 | 78 | 0.14 | 0.25 | 9 |
| 6 | Male | 42 | 83 | 0.15 | 0.28 | 9 |
| 7 | Female | 33 | 63 | 0.14 | 0.25 | 10 |
| 8 | Female | 31 | 68 | 0.15 | 0.28 | 10 |
| 9 | Female | 24 | 75 | 0.13 | 0.25 | 10 |
| 10 | Female | 55 | 62 | 0.13 | 0.25 | 8 |

Conclusion—On the basis of the results shown in the comparative studies given in Examples 9 and 10 hereinafter, the results are expected to indicate that the improved composition of the invention (Composition 1) is an especially effective combination to prevent or ameliorate the tested hangover symptoms, despite being substantially free from fructose, added caffeine and liquorice root.

### Example 2: Comparison of Prior Art (500 ml Fructose (90 g) and 200 ml Non-Fructose) Formulations with a Low-Volume Formulation of the Invention (50 ml)

One of the issues with the prior compositions, as described in US patent specification no. 6936283 is that the volume of the drink required to be consumed is relatively high: Comparative Composition A was consumed as a 500 ml drink; this is due to its high (90 g) fructose content. The prior US patent also describes a formulation (Comparative Composition B) that does not contain fructose and has a volume of 200 ml.

Therefore, in the prior art, the Clinical Study Protocol was carried out, wherein the test composition was Comparative Composition B (defined below); the given time was 30 minutes; and the given volume was reduced to 200 ml:

Comparative Composition B—A prior art composition was prepared in like manner to Comparative Composition A but instead comprising: 5 mg caffeine, 85 mg Guarana caffeine, 10 mg Yerba mate, 360 mg Eleutherococcus senticosus, 70 mg Panax Ginseng, 360 mg Glycyrrhiza glabra (Liquorice Root), 100 mg Ginkgo biloba and 1 g ginger. No fructose was added to the ingredients.

Results (prior art)—When taken 30 minutes after alcohol consumption, this prior art composition was found to accelerate alcohol degradation by an average of 60% as shown in Table 3.

The results indicate the contribution of fructose to the acceleration of alcohol degradation in the 500 ml Composition A to be approximately 20-25%.

TABLE 3
The reduced effect of a [200 ml, aqueous] non-fructose-containing
prior art composition on alcohol metabolism and hangover symptoms
Alcohol metabolism (%/hour)

| | | | | | Prior Art |
|---|---|---|---|---|---|
| Individual | Sex | Age | Weight (kg) | Baseline | Composition B |
| 1 | Male | 40 | 70 | 0.15 | 0.25 |
| 2 | Male | 35 | 95 | 0.15 | 0.23 |
| 3 | Male | 29 | 85 | 0.16 | 0.25 |
| 4 | Male | 57 | 115 | 0.17 | 0.26 |
| 5 | Male | 30 | 78 | 0.14 | 0.22 |
| 6 | Male | 42 | 83 | 0.15 | 0.24 |
| 7 | Female | 33 | 63 | 0.14 | 0.22 |
| 8 | Female | 31 | 68 | 0.15 | 0.25 |
| 9 | Female | 24 | 75 | 0.13 | 0.20 |
| 10 | Female | 55 | 62 | 0.13 | 0.21 |

Conclusion (prior art)—However, although this non-fructose-containing prior art formulation (Comparative Composition B) provided the benefit of a significantly reduced volume (200 ml v 500 ml) to consume compared to the fructose-containing prior art formulation, the total effect on alcohol degradation is somewhat reduced.

Furthermore, the volume was only reduced to 200 ml, which may still be considered substantial and smaller volumes are more preferred.

The present inventors sought to overcome these issues. Therefore, an experiment was carried out to test whether it is possible to prepare a very significantly smaller (50 ml) volume of a novel composition of the present invention, namely Compositions 2A and 2B, as defined below. Thereafter, both Compositions 2A and 2B may be compared with Comparative Composition B (prior art) using the Clinical Study Protocol (given time 30 minutes and given volume 50 ml):

Composition 2A of the invention—250 mg Ilex paraguariensis, 53 mg Panax Ginseng, 27 mg Eleutherococcus senticosus, 83 mg Zingiberis officinalis, 190 mg sodium gluconate, 1800 mg potassium gluconate, 550 mg magnesium lactate and 5000 mg fructose were mixed together and water was added.

Composition 2B of the invention—A second composition of the invention (Composition 2B) can be prepared in which the 5000 mg fructose component in Composition 2A is replaced by 400 μg sulphoraphane.

Results (Compositions of the Invention)

Volume Test—It was surprisingly found that a drink comprising Composition 2A of the invention could be prepared using a relatively low quantity of water, which produced a 50 ml drink that was nevertheless palatable, despite the relatively low quantity of fructose, compared to Composition A of the prior art (as defined in Example 1, above).

It is expected, on the basis of the result above and those given in Example 9 hereinafter, that the drink containing sulphoraphane instead of fructose will also be preparable to a volume as low as 50 ml. This is because Composition 9B in Example 9 shows that a similar composition to Composition 2B was able to be prepared to a 50 ml volume, despite additionally comprising 4 g fructose and also caffeine ingredients.

Study Protocol Test—In the light of the results given in Examples 9 and 10 hereinafter, when the study protocol is carried out on the new, improved low-volume (50 ml) formulations (Compositions 2A and 2B) according to the present invention, it is expected that, although some reduction in acceleration of alcohol degradation will be experienced, compared to the prior art formulations (Comparative Compositions A and B), nevertheless, the anti-hangover effect will still be satisfactory, despite the absence of anti-hangover amounts of fructose and some other ingredients, such as added caffeine, liquorice and Ginkgo biloba.

### Examples 3-8: Further Compositions of the Invention

Using the preparative method hereinbefore described, the following compositions were prepared:

### Example 3: Aqueous 50 ml Drink Composition Comprising Essential Herbs, Electrolytes and Low Fructose (Composition 3)

| Ingredient | Per serving (mg) |
|---|---|
| Ilex paraguariensis | 250* |
| Panax Ginseng | 53* |
| Eleutherococcus | 27* |
| senticosus | |
| Zingiberis officinalis | 83* |
| Sodium Gluconate | 190 |
| Potasium Gluconate | 1800 |
| Magnesium Lactate | 550 |
| Fructose | 5000 |

*rounded to the nearest integer and based on a stabilized form of the pure extracts.

Other minor ingredients:ethanol (96%), potassium sorbate, glycerol, citric acid monohydrate, Polysorbate 20 and aroma.

Water, q.s. to 50 ml.

### Example 4: Aqueous 50 ml Drink Composition Comprising Essential Herbs, Electrolytes, Low Fructose, and Additional Active Ingredients (Composition 4)

| Ingredient | Per serving (mg) |
|---|---|
| Caffeine | 0.5 |
| Guarana caffeine | 0.5 |
| Ilex paraguariensis | 200 |
| Panax Ginseng | 40 |
| Eleutherococcus | 20 |
| senticosus | |
| Zingiberis officinalis | 83 |
| Glycyrrhiza glabra | 10 |
| Sodium Gluconate | 190 |
| Potasium Gluconate | 1800 |
| Magnesium Lactate | 550 |
| Fructose | 5000 |

Other minor ingredients:ethanol (96%), potassium sorbate, glycerol, citric acid monohydrate, Polysorbate 20 and aroma.

Water, q.s. to 50 ml.

### Example 5: Composition of Essential Herbs—1 (Composition 5)

| | Per 50 ml dose |
|---|---|
| Ingredient | (mg) |
| Ilex paraguariensis | 200* |
| Panax Ginseng | 40* |
| Eleutherococcus | 20* |
| senticosus | |
| Zingiberis officinalis | 62.5* |

*based on water-soluble pure extracts

The four herbs were mixed together to form a composition of essential herbs, then electrolytes added as described hereinbefore to prepare Composition 5 of the invention. The resulting Composition 5 may be further processed by adding formulating and palatability ingredients, and optionally formulating into a capsule or dissolved in aqueous liquid to form a drink.

### Example 6: Composition of Essential Herbs—2 (Composition 6)

| | One-shot |
|---|---|
| Ingredient | preparation (mg) |
| Ilex paraguariensis | 600 |
| Panax Ginseng | 200 |
| Eleutherococcus | 500 |
| senticosus | |
| Zingiberis officinalis | 500 |

The four herbs were mixed together to form a composition of essential herbs, then water, electrolytes, palatability and formulating ingredients plus more water added as described hereinbefore to prepare Composition 6 of the invention as a 50 ml one-shot preparation.

### Example 7: Aqueous Drink (500 ml) Composition Comprising Essential Herbs, Electrolytes and Sulphoraphane (Composition 7)

A 500 ml aqueous formulation of the invention comprises: 250 mg Ilex paraguariensis, 53 mg Panax Ginseng, 27 mg Eleutherococcus senticosus, 83 mg Zingiberis officinalis, 400 mcg sulphoraphane, 300 mg sodium gluconate, 3000 mg potassium gluconate and 1100 mg magnesium lactate; and water (q.s. to 500 ml).

### Example 8: Aqueous Drink Composition Comprising Essential Herbs, Electrolytes, and Sulphoraphane

A 50 ml aqueous formulation of the invention comprises: 250 mg Ilex paraguariensis, 53 mg Panax Ginseng, 27 mg Eleutherococcus senticosus, 83 mg Zingiberis officinalis, 190 mg sodium gluconate, 1800 mg potassium gluconate, 550 mg magnesium lactate and 400 μg sulphoraphane; and water (q.s. to 50 ml).

### Example 9: Clinical Trial Demonstrating Beneficial Effect of Sulphoraphane

The purpose of this study was to compare the effect on alcohol metabolism and selected hangover symptoms of a formulation of the invention (Composition 9A) with the same formulation having added sulphoraphane (Composition 9B):

Composition 9A of the invention

| | (mg) per 50 ml |
|---|---|
| Active Ingredient | serving/dose |
| Caffeine | 0.5 |
| Guarana | 0.5 |
| Ilex paraguariensis | 250 |
| Panax Ginseng | 53 |
| Eleutherococcus senticosus | 27 |
| Zingiberis officinalis | 83 |
| Glycyrrhiza glabra | 10 |
| Sodium gluconate | 190 |
| Potasium gluconate | 1800 |
| Magnesium lactate | 550 |
| Fructose | 4000 |

Other ingredients:ethanol (final concentration in solution 0.67%), sodium benzoate, phosphoric acid, polydextrose, glycerol and aroma.

Composition 9B of the invention

| | (mg) per 50 ml |
|---|---|
| Active Ingredient | serving/dose |
| Caffeine | 0.5 |
| Guarana | 0.5 |
| Ilex paraguariensis | 250 |
| Panax Ginseng | 53 |
| Eleutherococcus senticosus | 27 |
| Zingiberis officinalis | 83 |
| Glycyrrhiza glabra | 10 |
| Sodium gluconate | 190 |
| Potasium gluconate | 1800 |
| Magnesium lactate | 550 |
| Fructose | 4000 |
| Sulphoraphane | 400 μg** |

**Incorporated as 8 mg sulphoraphane (5%) (equivalent to 400 μg active sulphoraphane), available from Nutra Green Biotechnology Co., Ltd.

Other ingredients:ethanol (final concentration in solution 0.67%), sodium benzoate, phosphoric acid, polydextrose, glycerol and aroma

Material and Methods—The study was a double-blind controlled two-group (Composition 9A vs. Composition 9B) within-subjects design in which participants consumed alcohol and Composition 9A or Composition 9B on the first day, and alcohol and Composition 9B or Composition 9A on the second day (counterbalanced order). Effectively, the method was as for the Clinical Study Protocol used in Examples 1 and 2 above, but with updated (expanded) hangover parameters, as detailed further below.

Participants were 11 healthy young women and men, aged from 27 to 36 years and weighing in the range of from 70 to 130 kg.

Inclusion criteria:
a. Healthy women and men between 20-40 years.
b. Non-Smoking.
c. No chronic diseases
d. Have previously experienced hangover symptoms as a result of alcohol intake.
e. The study only included participants who had consumed an equivalent amount of alcohol that was given in the study during the past few months.

Exclusion criteria
a. Smokers
b. Had a chronic condition or are on any medication.
c. Had drunk more than 5 units of alcohol in the last 5 days before the studies.
d. Consumption of alcohol over the 7-day period between the two test days.

Control design—Each participant was served alcohol (vodka 37.5%) on both days (day 1 and day 8) of the study. The amount of alcohol was calculated based on the participants' body weight. Participants acted as their own control in the following way:

Conduct of the study (both days)—Participants met up, and were instructed to draw a participant number. Which version of the formulation (Composition 9A or Composition 9B) the participants received on day 1 and day 8 was determined on the basis of this number. This procedure was followed both days, to ensure a true double-blind design and to ensure the participants' total anonymity in the report.

All participants were then served a standard meal, consisting of a baguette with ham and cheese and 1-2 glass(es) of milk (low-fat milk, 1.3% fat) or orange juice.

Alcohol administration started 30 minutes after the meal, and ended 60 minutes later. The alcohol was consumed in the form of 37.5% vodka (Boris Jelzin Vodka, Vinmonoplolet). The alcohol was mixed with caffeine-free beverages.

Evidence from previous experimental studies indicate that an alcohol dosage which provides a peak BAC (blood alcohol concentration) at approximately 1.1-1.2‰, is effective in inducing hangover. At the same time, such a BAC is usually not associated with other unwanted side effects, such as antisocial behaviour, vomiting, etc. The amount of alcohol needed to produce the desired BAC was calculated based on body weight as shown in Table 1 below. The amounts in the table were based on calculations from the Department of Public Health and The Norwegian Directorate of Health and Social Affairs, where the alcohol concentration is based on which body fluid volume the alcohol can be distributed. The average value for the latter is 68% of body weight.

TABLE 4
Amount (cl) of vodka 37.5% per body weight needed to
reach a BAC of 1.1-1.2%

| | | BAC (Blood Alcohol Concentration) |
|---|---|---|
| | Amount of | in % after ending |
| Weight (kg) | 37.5% vodka (ml) | alcohol consumption (high/low) |
| 70 | 205 | 1.17/1.11 |
| 75 | 220 | 1.18/1.12 |
| 80 | 235 | 1.18/1.12 |
| 85 | 250 | 1.18/1.12 |
| 90 | 270 | 1.21/1.15 |
| 95 | 285 | 1.21/1.15 |
| 100 | 300 | 1.21/1.15 |
| 105 | 315 | 1.21/1.15 |
| 110 | 330 | 1.21/1.15 |
| 115 | 345 | 1.21/1.15 |
| 120 | 360 | 1.21/1.15 |
| 125 | 375 | 1.21/1.15 |
| 130 | 390 | 0001.21 1.15 |

After alcohol consumption had finished, each participant was given 50 ml of either Composition 9 or Composition 10, as defined above.

Measurement of breath samples—BAC was measured by breath samples taken with an Alkometer Lifeloc FC10 apparatus (https://www.lifelo.comc/fc10), once every 90 minutes. Each value was an average of two parallel measurements.

Hangover symptoms evaluation—Previous studies have shown that hangover symptoms are usually worse +/−one hour after the BAC has dropped to 0.00‰. In this study, the following parameters, based on the Hangover Research Group consensus published in Curr. Drug Abuse Rev. (2010) 3(2) 116-126, were used to evaluate the effect of Composition 9A and Composition 9B on hangover:
a. Overall hangover
b. Headache
c. Fatigue
d. Nausea

On both days (Day 1 and Day 8), the participants were asked to complete a form evaluating the effect of the given formula, where a score of 10 indicated no symptoms of the syndrome and score 1 indicated no effect at all.

Results

All participants completed both test days. No adverse effects were observed at max BAC, 1.1-1.2‰.

Effect on Alcohol Degradation

Baseline—About 90-98% of the alcohol consumed is broken down in the liver by the enzyme called Liver Alcohol Dehydrogenase (LADH).

All trial participants, except participant #4, had an average rate of alcohol degradation of between 0.13-0.18‰/hour, with an average of 0.15‰/hour (baseline). Participant #4 had an average alcohol degradation of 0.23‰/hour (baseline). This high rate indicates that the CytP450 and/or catalase enzyme is/are active in the breakdown of alcohol in this person.

Composition 9A

The average rate of alcohol degradation when consuming the formulation (Composition 9A) was 0.19‰/hour (Table 5). This is on average 26.67% higher compared to baseline. Participant #4 had an increase of 24% compared to its baseline.

TABLE 5
BAC (Blood Alcohol Concentration) with Formulation (Composition 9A)

| | Gender | | Decrease in BAC |
|---|---|---|---|
| | (Female (F)/ | | (%/hour) |
| Participant # | Male (M)) | Weight (kg) | After Composition 9A |
| 1 | M | 98 | 0.180 |
| 2 | M | 79 | 0.168 |
| 3 | M | 102 | 0.202 |
| 4 | M | 79 | 0.285 |
| 5 | M | 92 | 0.152 |
| 6 | M | 100 | 0.185 |
| 7 | M | 130 | 0.162 |
| 8 | F | 74 | 0.170 |
| 9 | M | 81 | 0.210 |
| 10 | M | 100 | 0.176 |
| 11 | F | 71 | 0.180 |

| | |
|---|---|
| Average decrease in BAC (%/hour) | 0.190 |

Composition 9B

The average rate of alcohol degradation when consuming the formulation containing sulphoraphane (Composition 9B) was 0.216‰/hour (Table 6). This is on average 43.5% higher compared to the baseline. The increase in alcohol degradation for participant #4 was approximately 100%. This increase indicates that also other enzymes than just LADH may be activated (see discussion).

TABLE 6
BAC (Blood Alcohol Concentration) with sulphoraphane-containing
formulation (Composition 9B)

| | | | Decrease in BAC |
|---|---|---|---|
| | | | (%/hour) after |
| Participant # | Gender (F/M) | Weight (kg) | Composition 9B |
| 1 | M | 98 | 0.182 |
| 2 | M | 79 | 0.204 |
| 3 | M | 102 | 0.203 |
| 4 | M | 79 | 0.465 |
| 5 | M | 92 | 0.182 |
| 6 | M | 100 | 0.188 |
| 7 | M | 130 | 0.170 |
| 8 | F | 74 | 0.184 |
| 9 | M | 81 | 0.230 |
| 10 | M | 100 | 0.186 |
| 11 | F | 71 | 0.188 |

| | |
|---|---|
| Average decrease in BAC (%/hour) | 0.217 |

Effect on Hangover Symptoms

The reported effects on selected alcohol induced hangover symptoms of Composition 9A and Composition 9B are summarized in Table 7, in which a score of 10 represents ‘zero’ symptoms and a score of 1 represents ‘very strong’ symptoms, as reported by the study participants:

TABLE 7
Effect against hangover of Composition 9A vs Composition 9B

| | Effect against hangover | | | |
|---|---|---|---|---|
| | symptoms with the | | Effect against hangover | |
| | formulation without | | symptoms with the | |
| | sulphoraphane | | sulphoraphane-containing | |
| Participant # | (Composition 9A) | | formulation (Composition 9B) | |
| 1 | General hangover | 10 | General hangover | 10 |
| | Headache | 10 | Headache | 10 |
| | Fatigue | 9 | Fatigue | 10 |
| | Nausea | 10 | Nausea | 10 |
| 2 | General hangover | 9 | General hangover | 10 |
| | Headache | 10 | Headache | 10 |
| | Fatigue | 9 | Fatigue | 10 |
| | Nausea | 10 | Nausea | 10 |
| 3 | General hangover | 4 | General hangover | 10 |
| | Headache | 10 | Headache | 10 |
| | Fatigue | 4 | Fatigue | 10 |
| | Nausea | 10 | Nausea | 10 |
| 4 | General hangover | 4 | General hangover | 8 |
| | Headache | 8 | Headache | 10 |
| | Fatigue | 3 | Fatigue | 8 |
| | Nausea | 2 | Nausea | 8 |
| 5 | General hangover | 9 | General hangover | 10 |
| | Headache | 10 | Headache | 10 |
| | Fatigue | 7 | Fatigue | 9 |
| | Nausea | 9 | Nausea | 9 |
| 6 | General hangover | 10 | General hangover | 10 |
| | Headache | 10 | Headache | 10 |
| | Fatigue | 10 | Fatigue | 10 |
| | Nausea | 10 | Nausea | 10 |
| 7 | General hangover | 7 | General hangover | 10 |
| | Headache | 10 | Headache | 10 |
| | Fatigue | 4 | Fatigue | 8 |
| | Nausea | 8 | Nausea | 10 |
| 8 | General hangover | 10 | General hangover | 10 |
| | Headache | 10 | Headache | 10 |
| | Fatigue | 10 | Fatigue | 10 |
| | Nausea | 10 | Nausea | 10 |
| 9 | General hangover | 10 | General hangover | 10 |
| | Headache | 10 | Headache | 10 |
| | Fatigue | 10 | Fatigue | 10 |
| | Nausea | 10 | Nausea | 10 |
| 10 | General hangover | 8 | General hangover | 10 |
| | Headache | 10 | Headache | 10 |
| | Fatigue | 4 | Fatigue | 10 |
| | Nausea | 10 | Nausea | 10 |
| 11 | General hangover | 10 | General hangover | 10 |
| | Headache | 10 | Headache | 10 |
| | Fatigue | 9 | Fatigue | 10 |
| | Nausea | 10 | Nausea | 10 |

In summary, although the average score for each effect of Composition 9A against hangover symptoms was generally high:
General hangover: 8.27
Headache: 9.81
Fatigue: 6.27
Nausea: 9.00
the scores were higher for all symptoms for those who had taken Composition 9B:
General hangover: 9.81
Headache: 10.00
Fatigue: 9.55
Nausea: 9.73.

The better effect of Composition 9B compared to Composition 9A was most pronounced for general hangover and fatigue. The results were consistent, as no better effect for Composition 9A compared to Composition 9B was reported by any of the participants for any symptoms.

Discussion:

Effect on Alcohol Breakdown

Although not wishing to be bound by theory, it is proposed that the mechanism behind Composition 9A's increase in alcohol metabolism is such that the Composition 9A forms an enzyme-NADH-Composition 9A tertiary complex which dissociates faster (k5) than the normal rate-determining E-NADH dissociation (k4).

The results from the study show an average increase of 44.6% in the rate of alcohol degradation, when participants were served the formulation (Composition 9B) containing sulphoraphane. This is more than 50% higher than the increase observed with Composition 9A.

These results were neither obvious nor expected, as previous research (Langeland, B T, et al. (1999) Metal binding properties of thiols; complexes with horse liver alcohol dehydrogenase. Comp Biochem Physiol B Biochem Mol Biol. 123, 155-62) has shown that sulphur- and especially thiol-compounds usually have a strong inhibitory effect on dehydrogenase enzymes like LADH.

The extra increase in alcohol degradation with Composition 9B compared to that with Composition 9A suggests that the sulphoraphane component in Composition 9B may bind to the LADH enzyme at a place/site where it is not competing with the other ingredients.

Instead, it contributes to the formation of a ternary E-NADH-Composition 9B complex that dissociates even faster than the ternary E-NADH-Composition 9A complex.

The significantly higher increase in alcohol degradation for participant #4 with Composition 9B further suggest that sulphoraphane also increases the activity of Cyt P450 and/or catalase enzyme, as these enzymes also can contribute to alcohol breakdown in individuals with chronic/high alcohol consumption.

Effect on Acetaldehyde Breakdown: ALDH Activity/Hangover Symptoms

Aldehyde dehydrogenase (ALDH) catalyses the critical step of ethanol metabolism, i.e. transformation of toxic acetaldehyde (which is 30 times more toxic than ethanol) to acetic acid

Many compounds containing sulphur (e.g. disulfiram) have been found to inhibit ALDH enzyme activity. The observed increased positive effects on selected hangover symptoms by the sulphoraphane-containing Composition 9B were therefore not obvious or could not have been predicted from an enzymatic point of view.

The results suggest that sulphoraphane, much in the same way as for LADH, may bind to a place/site at the ALDH enzyme where it increases the activity of the enzyme most probably by contributing to the formation of a complex dissociating faster than the rate-determining step of ALDH.

Another possible mechanism is that sulphoraphane and/or other components of Composition 9B, aid the body's own production of glutathione, which previously has been shown to partly alleviate some symptoms associated with alcohol induced hangover.

Conclusion

The results from the present study show that the formulation (Composition 9B) containing sulphoraphane has a significantly better effect than Composition 9A on both the rate of alcohol degradation in the body and against hangover symptoms.

The sulphoraphane content of the Composition 9B may also contribute to a better effect against “Asian flushing syndrome”.

Nevertheless, Composition 9A is a significant improvement over the prior art compositions as:

the significant reduction in the fructose ingested avoids both depleting phosphates in the liver cells and stressing the liver;

the reduced volume makes the composition light and pleasant to consume—it does not cause nausea (which is often the case with a 500 ml high-fructose drink); and

the diuretic effect of alcohol is counteracted by ingesting electrolytes.

### Example 10: Clinical Trial Demonstrating Beneficial Effect of High Yerba Mate and Added Electrolytes in Absence of High Fructose and Added Caffeine/Liquorice

The method of Example 9 was repeated, but this time to compare the 500 ml formulation of Comparative Composition A of the prior art (as defined in Example 1 of U.S. Pat. No. 6,936,283) with a 500 ml formulation of a Composition 10 of the invention comprising:

250 mg Ilex paraguariensis, 53 mg Panax Ginseng, 27 mg Eleutherococcus senticosus, 83 mg Zingiberis officinalis, 5000 mg fructose, 300 mg sodium gluconate, 3000 mg potassium gluconate and 1100 mg magnesium lactate.

The participants were all males, aged 27 to 34 years, having a bodyweight in the range of 80 to 114 kg.

The test parameters (rate of alcohol degradation and effect on hangover) were as described in Example 9.

Results

TABLE 8
Rate of alcohol degradation (%/hour)

| | | | Decrease in | |
|---|---|---|---|---|
| | | | BAC (%/hour) | Decrease in |
| | Gender | | Comparative | BAC (%/hour) |
| Participant # | (F/M) | Weight (kg) | Composition A | Composition 10 |
| 1 | M | 80 | 0.25 | 0.24 |
| 2 | M | 81 | 0.30 | 0.27 |
| 3 | M | 102 | 0.25 | 0.20 |
| 4 | M | 93 | 0.26 | 0.24 |
| 5 | M | 114 | 0.25 | 0.20 |
| 6 | M | 96 | 0.25 | 0.24 |
| 7 | M | 101 | 0.26 | 0.20 |
| 8 | M | 99 | 0.24 | 0.20 |

| | | |
|---|---|---|
| Rate (%/hour) all partcipants | 0.26 | 0.22 |

TABLE 9
Effect against hangover

| | Effect against hangover | | Effect against hangover | |
|---|---|---|---|---|
| | symptoms with Comparative | | symptoms with | |
| Participant # | Composition A | | Composition 10 | |
| 1 | General hangover | 8 | General hangover | 9 |
| | Headache | 8 | Headache | 10 |
| | Fatigue | 7 | Fatigue | 9 |
| | Nausea | 10 | Nausea | 10 |
| 2 | General hangover | 10 | General hangover | 10 |
| | Headache | 10 | Headache | 10 |
| | Fatigue | 10 | Fatigue | 10 |
| | Nausea | 10 | Nausea | 10 |
| 3 | General hangover | 7 | General hangover | 10 |
| | Headache | 6 | Headache | 10 |
| | Fatigue | 5 | Fatigue | 9 |
| | Nausea | 5 | Nausea | 10 |
| 4 | General hangover | 8 | General hangover | 9 |
| | Headache | 9 | Headache | 9 |
| | Fatigue | 6 | Fatigue | 9 |
| | Nausea | 8 | Nausea | 9 |
| 5 | General hangover | 9 | General hangover | 10 |
| | Headache | 7 | Headache | 8 |
| | Fatigue | 10 | Fatigue | 8 |
| | Nausea | 10 | Nausea | 10 |
| 6 | General hangover | 8 | General hangover | 10 |
| | Headache | 10 | Headache | 8 |
| | Fatigue | 5 | Fatigue | 10 |
| | Nausea | 10 | Nausea | 10 |
| 7 | General hangover | 7 | General hangover | 9 |
| | Headache | 9 | Headache | 9 |
| | Fatigue | 5 | Fatigue | 7 |
| | Nausea | 9 | Nausea | 10 |
| 8 | General hangover | 7 | General hangover | 10 |
| | Headache | 6 | Headache | 10 |
| | Fatigue | 2 | Fatigue | 7 |
| | Nausea | 6 | Nausea | 10 |

Average scores for the prior art Comparative Composition A effect against hangover symptoms;
General hangover: 8
Headache: 8.125
Fatigue: 6.25
Nausea: 8.5
Average scores for Composition 10 of the present invention;
General hangover: 9.625
Headache: 9.375
Fatigue: 8.625
Nausea: 9.875

It is also notable that the rate of alcohol metabolism was faster for the low-fructose Composition 10 than for the low-fructose composition of the invention used in Example 9 (Composition 9A), but which had a lower content of electrolytes. This suggests that the electrolytes themselves are capable of activating the LADH (and ALDH) enzyme.

The participants also found it very hard and unpleasant to consume the 500 ml high-fructose formulation of the prior art; yet, they found the 500 ml Composition 10 formulation of the invention very easy to consume. Moreover, the Composition 10 of the invention was able to be formulated as a low-volume (50 ml) drink, which was not possible for the prior art compositions.

Conclusion

Therefore, a composition of the invention (Composition 10) was found to product a satisfactory effect against hangover, despite being free from both caffeine and liquorice, and comprising only a sweetening amount of fructose.

In effect, improving on the prior art formulation by substantially increasing the proportion of Yerba mate in the essential herb mix (so that the Yerba mate is the major component of the essential her mix and no other herbs, such as G. glabra or Paullinia culpana, are necessary) and adding electrolytes means that other less desirable ingredients—or undesirable quantities of ingredients—can be avoided.

Furthermore, the composition of the invention (Composition 10) can be formulated as a 50 ml drink (or ‘shot’), with the advantage that consumers would not need to drink 500 ml of liquid in one dose.

The effect on the hangover symptoms was noticeably better with Composition 10 of the invention, compared to Comparative Example A of the prior art.

Although the alcohol metabolism was somewhat faster after consuming a high fructose content formulation (Comparative Example A) compared to a low-fructose formulation (Composition 10), the rate was still acceptable for—and did not adversely affect the anti-hangover performance of—the composition of the invention.

There was also some variation among the participants, which probably relates to differences between their plasma fructose concentrations.

Accordingly, addition of electrolytes to—together with significantly lowering (or excluding) fructose from—a composition comprising essential herbs, a major component of which is Yerba mate, provides unexpected advantages over previously-known herb-based compositions for treating or preventing or ameliorating hangover symptoms.

1-20. (canceled)

21. A composition for the promotion of alcohol and acetaldehyde degradation in a human, which composition comprises an anti-hangover effective amount of:
(a) essential herbs consisting of Ilex paraguarensis (Yerba mate), Eleutherococcus senticosus (Siberian ginseng), Panax ginseng and Zingiberis officinalis (ginger);
(b) one or more electrolyte(s); and
(c) sulphoraphane.

22. The composition as claimed in claim 21, wherein the electrolyte(s) is selected from one or more of: alkali and alkaline earth metal salts of organic acids, optionally sodium gluconate, potassium gluconate and/or magnesium lactate.

23. The composition as claimed in claim 21, wherein the essential herbs comprise at least 1%, optionally at least 10%, optionally at least 30%, Yerba mate, by dry weight of the essential herb component (a).

24. The composition as claimed in claim 21, wherein the essential herbs comprise at least 50% Yerba mate, by dry weight of the essential herb component.

25. The composition as claimed in claim 21, wherein the essential herb component (a) is present in an amount less than about 1000 mg, optionally less than about 600 mg, optionally in the range of 400-450 mg, per dose.

26. The composition as claimed in claim 21, comprising, by weight of dry powder, per dose:
i. 150-500 mg Ilex paraguariensis,
ii. 30-100 mg Panax ginseng,
iii. 20-50 mg Eleutherococcus senticosus,
iv. 50-150 mg Zingiberis officinalis,
v. 150-400 mg sodium gluconate,
vi. 1500-2500 mg potassium gluconate, and
vii. 500-1100, optionally 500-1000 mg, magnesium lactate.

27. The composition as claimed in claim 21 comprising, by percentage of the weight of the listed ingredients i-vii and sulphoraphane:
i. 0.5%-5.0% w/w Ilex paraguariensis,
ii. 0.05-2.0% w/w Eleutherococcus senticosus,
iii. 0.01-1.0% Panax ginseng,
iv. 0.3-3.0% Zingiberis officinalis,
v. 0.3-3.0% sodium gluconate,
vi. 5.0-30.0% potassium gluconate, and
vii. 3.0-10.0% magnesium lactate.

28. The composition as claimed in claim 21, wherein the sulphoraphane is present in an amount in the range of from 200 to 800 optionally 200-400 sulphoraphane.

29. The composition as claimed in claim 21, further comprising one or more additional ingredient(s) selected from:
(a) additional active ingredients; or
(b) inert additives, optionally sweeteners, flavourings, colouring agents, other vitamins, stabilisers, preservatives and solubilisers,
provided that any additional ingredient (a) or (b) is present in an amount that does not inhibit the activity of alcohol- and acetaldehyde-metabolizing enzymes.

30. The composition as claimed in claim 21, which is substantially free from one or more of:
(a) fructose in an anti-hangover-effective amount, optionally fructose in excess of 5 g per dose;
(b) Glycyrrhiza glabra (liquorice root); or
(c) added caffeine.

31. The composition as claimed in claim 21 in the form of a dry powder, a mixture, a liquid (optionally a beverage or drink or shot), a syrup, granules or granulates, tablets (optionally effervescent or melt tablets), any of which is optionally packaged together with instructions for use.

32. The composition as claimed in claim 21, further comprising an aqueous liquid (optionally tap water, still water, spring water, carbonated water, fruit juice), and optionally one or more flavouring and/or colouring agents.

33. The composition as claimed in claim 32, wherein the composition is in the form of a ready-to use beverage, drink or shot.

34. The composition as claimed in claim 33 in the form of a 50 ml drink.

35. A method of preparing a composition according to claim 21, which method comprises:
(a) mixing all the dry ingredients together to form a dry mixture;
(b) adding water to the mixture provided by step (a) in a ratio of in the range of from 1:2 to 2:4 of dry mixture:water;
(c) stirring the mixture provided by step (b) until homogenous to form a concentrate;
(d) adding the remaining ingredients, including the electrolytes, and any additional active ingredients and any inert ingredients; and
(e) optionally diluting the concentrate produced in step (d) until a desired volume is obtained; and, optionally,
(f) filling the product from step (e) into a suitable container, optionally a glass, aluminium (optionally coated) or plastics bottle; and, optionally,
(g) packaging the product from step (e) in association with instructions for its use by the consumer.

36. A method comprising utilizing a composition as claimed in claim 21, in the preparation of a treatment of, prevention of or remedy for a condition selected from:
(a) hangover or veisalgia;
(b) alcohol-induced headache, nausea, shaking or vertigo;
(c) Asian flushing syndrome;
(d) an accumulation of acetaldehyde;
(e) dehydration;
(f) acute alcohol poisoning;
(g) alcohol-induced cell or tissue damage; or
(h) toxicity associated with disulfiram.

37. A method for the treatment, prevention or amelioration of hangover symptoms in a subject, which method comprises administration to the subject of an effective amount of a composition as claimed in claim 21.

38. The composition as claimed in claim 21, comprising, by weight of the total dry ingredients:
(a) as essential herbs: Yerba mate (0.5-5.0%, optionally 2.96%), Eleutherococcus senticosus (0.05-2.0%, optionally 0.32%), Panax ginseng (0.01-1.0%, optionally 0.62%), ginger (0.3-3.0%, optionally 0.98%); and
(b) (0.3-3.0%, optionally 2.24%) sodium gluconate, (5.0-30.0%, optionally 21.20-22.0%) potassium gluconate, (3.0-10.0%, optionally 6.48%) magnesium lactate, or the equivalent amounts of the Na, K and Mg ions when using alternative salts.
